(19) United States
(12) Patent Application Publication
(10) Pub. No.: US 2013/0226002 A1
(43) Pub. Date: Aug. 29, 2013

(54) ULTRASOUND DIAGNOSTIC DEVICE

(71) Applicant: FUJIFILM Corporation, (US)

(72) Inventor: Yukiya MIYACHI, Kanagawa (JP)

(73) Assignee: FUJIFILM Corporation, Tokyo (JP)

(21) Appl. No.: 13/857,078

(22) Filed: Apr. 4, 2013

Related U.S. Application Data
(63) Continuation of application No. PCT/JP2011/070374, filed on Sep. 7, 2011.

(30) Foreign Application Priority Data
Oct. 13, 2010 (JP) 2010-230525

(51) Int. Cl. A61B 8/15

(52) U.S. Cl. CPC A61B 8/15
USPC 600/448

(57) ABSTRACT

An ultrasound diagnostic apparatus comprises an ultrasound probe including a transducer array for transmitting and receiving ultrasound; a diagnostic apparatus body for generating ultrasound images; a communications cable connecting the ultrasound probe and the apparatus body with each other; an apparatus body-side connector for connecting one end of the communications cable with the diagnostic apparatus body; analog-to-digital converters for processing reception signals from the transducer array; an electrical-to-optical conversion unit for converting the processed reception signals into optical signals; an optical fiber provided in the communications cable in order to transmit the reception signals as optical signals; and an optical-to-electrical conversion unit for converting the transmitted reception signals into electric signals. The diagnostic apparatus body generates an ultrasound image based on the reception signals as converted by the optical-to-electrical conversion unit into electric signals.

### BACKGROUND OF THE INVENTION

The present invention relates to ultrasound diagnostic apparatus, and is particularly directed to an ultrasound diagnostic apparatus adapted to transmit a reception signal from an ultrasound probe to a diagnostic apparatus body through an optical fiber.

In the medical field, ultrasound diagnostic apparatus employing ultrasound images have already been put to practical use. A typical ultrasound diagnostic apparatus for medical use has an ultrasound probe with a transducer array built therein and an apparatus body connected with the ultrasound probe, and generates an ultrasound image by transmitting ultrasound from the ultrasound probe toward a subject, receiving an ultrasonic echo from the subject on the ultrasound probe, and electrically processing a reception signal corresponding to the received echo in the apparatus body.

In recent years, attempts are being made to conduct ultrasonic diagnosis on various regions of a subject, and diverse ultrasound probes have been developed for different applications. In addition, two-dimensional array-type ultrasound probes capable of omnidirectional change in focal position of an ultrasonic beam are in the process of development as an ultrasound probe allowing three-dimensional ultrasound images. Reception signals transmitted from an ultrasound probe will carry increased information with such developments as above, so that the transmission of reception signals needs to be made faster.

In this regard, JP 2010-42042 A, for instance, discloses the ultrasound diagnostic apparatus in which an optical fiber is provided in a communications cable connecting between an ultrasound probe and a diagnostic apparatus body, and a reception signal from the ultrasound probe is converted into an optical signal and transmitted by the optical fiber.

### SUMMARY OF THE INVENTION

In the ultrasound diagnostic apparatus of JP 2010-42042 A, reception signals are transmitted faster by the use of optical signals having a wider transmission band than electric signals. With the communications cable with the optical fiber provided therein and the diagnostic apparatus body being integrally connected with each other, however, higher costs are required if an ultrasound probe using optical signals for the transmission of reception signals or an ultrasound probe using electric signals for the transmission of reception signals should be selected appropriately to the application of interest because it is necessary to provide diagnostic apparatus bodies corresponding to the two probes, respectively.

The present invention has been made in order to solve the above problem with the prior art, aiming at providing an ultrasound diagnostic apparatus allowing the use of the ultrasound probes transmitting reception signals as optical signals and as electric signals that can each be connected with a diagnostic apparatus body in a detachable manner.

The ultrasound diagnostic apparatus according to the present invention is an ultrasound diagnostic apparatus having an ultrasound probe and a diagnostic apparatus body connected with each other through a communications cable, with the ultrasound probe containing a transducer array from which an ultrasonic beam is transmitted toward a subject, and the diagnostic apparatus body generating an ultrasound image based on reception signals outputted from the transducer array of the ultrasound probe that has received an ultrasonic echo from the subject, characterized in that one end of the communications cable is detachably connected with the diagnostic apparatus body through an apparatus body-side connector, and the ultrasound diagnostic apparatus comprises: an electrical-to-optical conversion means adapted to convert the reception signals having been processed by a plurality of analog-to-digital converters connected with the transducer array of the ultrasound probe into optical signals; an optical fiber provided in the communications cable and adapted to transmit the reception signals converted by the electrical-to-optical conversion means into the optical signals; and an optical-to-electrical conversion means contained in the apparatus body-side connector and adapted to convert the reception signals transmitted by the optical fiber as the optical signals into electric signals.

The ultrasound diagnostic apparatus as above may further comprise: a parallel-to-serial converter connected between the plurality of analog-to-digital converters and the electrical-to-optical conversion means in the ultrasound probe, and adapted to subject the reception signals from the plurality of analog-to-digital converters to conversion from parallel data into serial data and then transmit them to the electrical-to-optical conversion means; and a serial-to-parallel converter connected downstream of the optical-to-electrical conversion means in the apparatus body-side connector, and adapted to subject the reception signals from the optical-to-electrical conversion means to conversion from serial data into parallel data and then transmit them to the diagnostic apparatus body.

The electrical-to-optical conversion means may include a plurality of electrical-to-optical converters corresponding to the plurality of analog-to-digital converters, and the optical-to-electrical conversion means may include a plurality of optical-to-electrical converters corresponding to the electrical-to-optical converters. The ultrasound diagnostic apparatus may further comprise: an optical coupler adapted to combine the optical signals resulting from conversion by the plurality of electrical-to-optical converters into composite optical signals and transmit the composite optical signals to the optical fiber; and a wavelength division-type optical waveguide adapted to divide the composite optical signals transmitted via the optical fiber into optical signals with different wavelengths and feed the optical signals with different wavelengths to the plurality of optical-to-electrical converters, respectively.

The apparatus body-side connector preferably receives one end of the optical fiber by a specified length along one face of a connector board, and has connector pins uprightly provided at another face of the connector board. On one face of the connector board, a cable connector for a signal line dedicated to transmission signals, the optical-to-electrical conversion means, and an amplifier may be mounted along with the optical fiber.

It is also possible that the apparatus body-side connector receives one end of the optical fiber by a specified length along one face of a connector board, and has connector pins uprightly provided at another face of the connector board, and, on one face of the connector board, a cable connector for a signal line dedicated to transmission signals, the optical-to-electrical conversion means, an amplifier, and the serial-to-parallel converter are mounted along with the optical fiber.

The communications cable may detachably be connected at its one end with the ultrasound probe through a first optical fiber connector, and at the other end with the apparatus body-side connector through a second optical fiber connector.

According to the present invention, a communications cable with an optical fiber provided therein and a diagnostic apparatus body are detachably connected with each other through a connector containing an optical-to-electrical conversion means, which allows both ultrasound probes transmitting reception signals as optical signals and as electric signals, respectively, to be detachably connected with the diagnostic apparatus body upon use.

### BRIEF DESCRIPTION OF THE DRAWINGS

In the accompanying drawings:

FIG. 1 is a block diagram illustrating the ultrasound diagnostic apparatus according to Embodiment 1 of the present invention;

FIG. 2 is a block diagram illustrating the ultrasound diagnostic apparatus according to Embodiment 2;

FIG. 3 is a block diagram illustrating the ultrasound diagnostic apparatus according to Embodiment 3;

FIG. 4 is a block diagram illustrating the ultrasound diagnostic apparatus according to Embodiment 4;

FIG. 5 is a diagram showing the configuration of the ultrasound diagnostic apparatus according to Embodiment 5;

FIG. 6 is a plan/sectional view of the connector used in Embodiment 5, showing the configuration of the connector; and

FIG. 7 is a lateral/sectional view of the connector used in Embodiment 5, showing the configuration of the connector.

### DETAILED DESCRIPTION OF THE INVENTION

In the following, embodiments of the present invention will be described in reference to the accompanying drawings.

### Embodiment 1

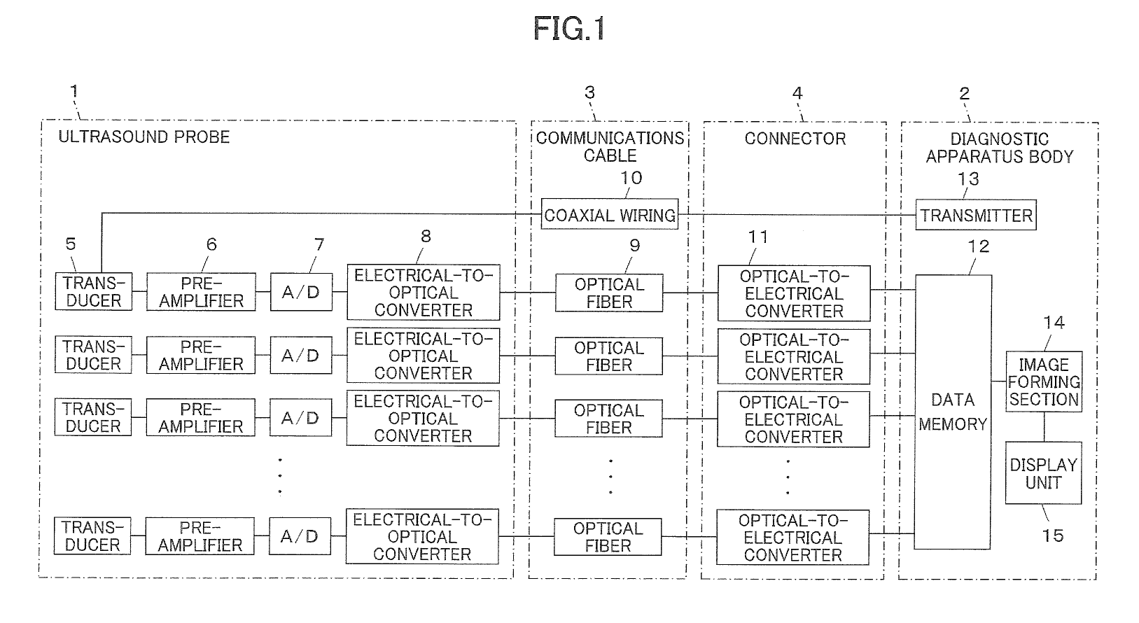

FIG. 1 illustrates the configuration of the ultrasound diagnostic apparatus according to Embodiment 1 of the present invention. The ultrasound diagnostic apparatus is comprised of an ultrasound probe 1, a diagnostic apparatus body 2 adapted to generate ultrasound images, a communications cable 3 connected with the ultrasound probe 1, and a connector 4 detachably connecting the communications cable 3 and the diagnostic apparatus body 2 with each other.

The ultrasound probe 1 has a one- or two-dimensional transducer array 5 consisting of a plurality of ultrasound transducers, a plurality of preamplifiers 6 connected correspondingly to the transducer array 5, and electrical-to-optical converters 8 connected with the preamplifiers 6 through analog-to-digital (A/D) converters 7, respectively. The ultrasound probe 1 includes a communications line for transmitting driving signals to the transducer array 5.

The transducers constituting the transducer array 5 transmit ultrasonic waves in accordance with driving signals fed through the communications line connected with the communications cable 3 and receive ultrasonic echos from a subject so as to output reception signals. Each transducer is comprised of a vibrator having a piezoelectric body and electrodes formed at both ends of the piezoelectric body, with the piezoelectric body being a piezoelectric element composed of a piezoelectric ceramic typified by PZT (lead zirconate titanate), a piezoelectric polymer typified by PVDF (polyvinylidene fluoride), or the like.

If a pulsed voltage or a continuous wave voltage is applied across the electrodes of the vibrator as above, the piezoelectric body expands and contracts, and an ultrasonic wave in pulsed form or continuous wave form is generated from the vibrator. Ultrasonic waves generated from individual vibrators are synthesized into an ultrasonic beam. In addition, each vibrator expands and contracts during the reception of propagating ultrasonic wave to generate an electric signal, which is outputted as a reception signal representing the reception of an ultrasonic wave. Input of a driving signal into or output of a reception signal from each vibrator is carried out by connecting the relevant vibrator selectively to the communications line for transmitting driving signals or to the corresponding preamplifier 6 through a transmission/reception selector switch not shown.

The preamplifiers 6 amplify reception signals outputted from the transducers in individual channels of the transducer array 5, respectively. The transducer array 5 has a specified frequency band and a specified driving voltage, and those preamplifiers with a frequency band corresponding to that of the transducer array 5 are used as the preamplifiers 6.

The A/D converters 7 digitize the reception signals as amplified by the preamplifiers 6, respectively. The reception signals as digitized by the A/D converters 7 are fed to the electrical-to-optical converters 8.

The electrical-to-optical converters 8 are adapted to convert a reception signal fed thereto as an electric signal into an optical signal by, for instance, using a semiconductor laser as a light source and modulating the intensity of an optical signal from the light source in response to the electric signal.

The communications cable 3 has a plurality of optical fibers 9 connected to the electrical-to-optical converters 8 of the ultrasound probe 1, respectively, with the reception signals as converted by the electrical-to-optical converters 8 into optical signals being transmitted via the optical fibers 9. The communications cable 3 also has a coaxial wiring 10, via which driving signals are transmitted to the transducer array 5.

The connector 4 has optical-to-electrical converters 11 connected with the optical fibers 9 of the communications cable 3, respectively, and includes a communications line connected to the coaxial wiring 10 of the communications cable 3. The optical-to-electrical converters 11 receive the optical signals as transmitted by the optical fibers 9, so as to convert them into electric signals.

The diagnostic apparatus body 2 has a data memory 12 and a transmitter 13, with the former being connected with the optical-to-electrical converters 11 of the connector 4 and the latter being connected to the coaxial wiring 10 of the communications cable 3 through the connector 4. The data memory 12 is connected to a display unit 15 through an image forming section 14.

The data memory 12 sequentially stores, as reception data, the reception signals as converted by the optical-to-electrical converters 11 into electric signals.

The image forming section 14 conducts reception focusing on the reception data as stored in the data memory 12 so as to generate an image signal representing an ultrasound diagnostic image, such as the B mode image signal as a tomographic image information on a tissue in the subject's body.

The display unit 15, as being adapted to display an ultrasound diagnostic image based on image signals generated by the image forming section 14, includes a display device such as an LCD.

The transmitter 13 is connected to the transducer array 5 of the ultrasound probe 1 through the coaxial wiring 10. The transmitter 13 includes a plurality of pullers, for instance, and feeds the transducers of the transducer array 5 with their respective driving signals having delay amounts modified so that ultrasonic waves transmitted from the transducer array 5 may be formed into a broad ultrasonic beam covering the area of a tissue in the subject's body.

In Embodiment 1, the communications cable 3 with the optical fibers 9 and the diagnostic apparatus body 2 can detachably be connected with each other through the use of the connector 4.

The following description is made on the operation of Embodiment 1.

Initially, driving signals are transmitted from the transmitter 13 of the diagnostic apparatus body 2 and fed to the transducer array 5 of the ultrasound probe 1 via the coaxial wiring 10 of the communications cable 3 connected with the transmitter 13 through the connector 4. Ultrasonic waves are transmitted from the transducers constituting the transducer array 5 in accordance with the driving signals as fed from the transmitter 13.

Then, the transducer array 5 is disconnected from the transmitter 13 before being connected to the preamplifiers 6, so that the reception signals as outputted from the transducers of the transducer array 5 that have received ultrasonic echos from a subject are inputted into the preamplifiers 6. The reception signals are amplified by the amplifiers 6 and digitized by the A/D converters 7, then fed to the electrical-to-optical converters 8 where they are converted into optical signals. The reception signals as converted into optical signals are transmitted to the optical-to-electrical converters 11 of the connector 4 via the optical fibers 9. The reception signals as inputted into the optical-to-electrical converters 11 of the connector 4 are converted into electric signals and outputted from the optical-to-electrical converters 11 to the data memory 12 of the diagnostic apparatus body 2.

With the optical-to-electrical converters 11 being thus provided in the connector 4 connecting the optical fibers 9 and the diagnostic apparatus body 2 together, it is no longer required of the diagnostic apparatus body 2 to process optical signals, so that the diagnostic apparatus body 2 is able to be connected not only with an ultrasound probe connected with the communications cable which transmits electric signals but an ultrasound probe connected with the communications cable which transmits optical signals.

The reception signals as outputted from the optical-to-electrical converters 11 are sequentially stored in the data memory 12 as reception data. Subsequently, reception data stored in the data memory 12 is inputted into the image forming section 14, where an image signal representing an ultrasound diagnostic image is generated. Based on the image signal thus generated, an ultrasound diagnostic image is displayed on the display unit 15.

According to Embodiment 1, various ultrasound probes are connectible to the diagnostic apparatus body as appropriate to different applications merely by using the connector 4 to change the ultrasound probe 1 connected with the communications cable 3 to another such probe.

The optical fibers 9 may be glass optical fibers, plastic optical fibers, or multicore fibers. The light source to be used for the optical fibers 9 is exemplified by a VCSEL, light source of surface emitting type capable of high-speed operation even at a low voltage, while available photoreceivers include a planar photoreceiver with a large area, easy to connect and, moreover, capable of fast response, such as an MSM PD and a lateral PIN PD.

### Embodiment 2

The optical fibers 9 of the communications cable 3 used in Embodiment 1 are corresponding in number to the transducer array 5 of the ultrasound probe 1, to which the present invention is not limited. The number of optical fibers can be reduced using a parallel-to-serial converter.

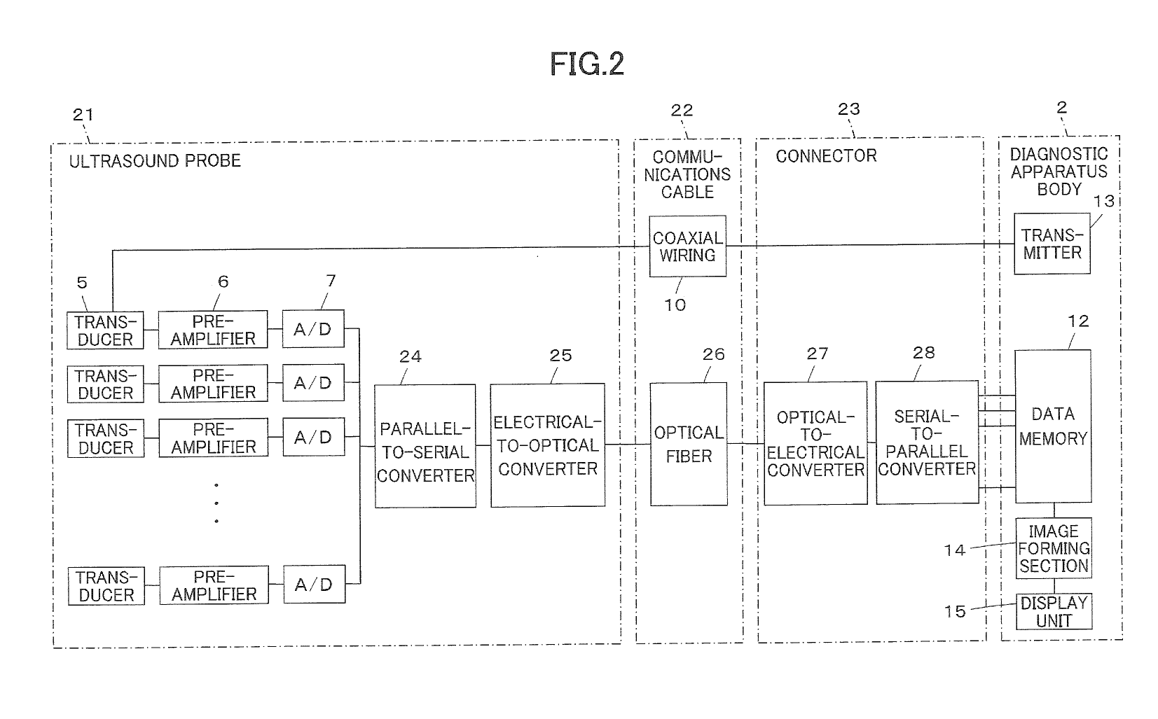

As shown in FIG. 2, for instance, a communications cable used in an ultrasound diagnostic apparatus may have a single optical fiber provided therein. In the ultrasound diagnostic apparatus of FIG. 2, the ultrasound probe 1, the communications cable 3 and the connector 4 in Embodiment 1 as shown in FIG. 1 are replaced by an ultrasound probe 21, a communications cable 22 and a connector 23, respectively. The ultrasound probe 21 does not have the electrical-to-optical converters 8 of the ultrasound probe 1 in Embodiment 1 but a parallel-to-serial converter 24 connected with the A/D converters 7, with the parallel-to-serial converter 24 being also connected to an electrical-to-optical converter 25. The communications cable 22 has a single optical fiber 26 connected with the electrical-to-optical converter 25 of the ultrasound probe 21, instead of the optical fibers 9 of the communications cable 3 in Embodiment 1. The connector 23 has an optical-to-electrical converter 27 connected with the optical fiber 26, instead of the optical-to-electrical converters 11 of the connector 4 in Embodiment 1, with the optical-to-electrical converter 27 being also connected to a serial-to-parallel converter 28. The serial-to-parallel converter 28 of the connector 23 is connected to the data memory 12 of the diagnostic apparatus body 2.

The parallel-to-serial converter 24 converts the parallel reception signals as digitized by the A/D converters 7 into serial reception signals. The serial-to-parallel converter 28 converts the serial reception signals as outputted from the optical-to-electrical converter 27 into parallel reception signals.

Similar to Embodiment 1, the reception signals as outputted from the transducer array 5 are amplified by the preamplifiers 6 and digitized by the A/D converters 7. The parallel reception signals thus digitized are converted by the parallel-to-serial converter 24 into serial reception signals, then outputted therefrom to the electrical-to-optical converter 25 which converts the serial reception signals into optical signals. The serial reception signals as converted into optical signals are transmitted from the electrical-to-optical converter 25 to the optical-to-electrical converter 27 via the single optical fiber 26 contained in the communications cable 22, and subjected by the optical-to-electrical converter 27 to conversion from optical signals into electric ones. The serial reception signals as converted into electric signals are outputted from the optical-to-electrical converter 27 to the serial-to-parallel converter 28, and further converted by the serial-to-parallel converter 28 into parallel reception signals. The parallel reception signals as outputted from the serial-to-parallel converter 28 are sequentially stored in the data memory 12 of the diagnostic apparatus body 2 as reception data.

According to Embodiment 2, temperature rise in the ultrasound probe 21 is suppressed because the ultrasound probe 21 is reduced in number of electrical-to-optical converters provided therein. In addition, the communications cable is reduced in thickness so as to make the cable easier to handle.

It is also possible to divide a plurality of A/D converters 7 into two or more groups and connect each group of A/D converters 7 to one optical fiber 26 through a parallel-to-serial converter 24 and an electrical-to-optical converter 25, so as to transmit optical signals group by group. Such configuration allows the communications cable 22 to be changed in number of optical fibers 26 therein depending on the situation.

### Embodiment 3

The optical fibers 9 of the communications cable 3 used in Embodiment 1 may also be reduced in number by using an optical coupler.

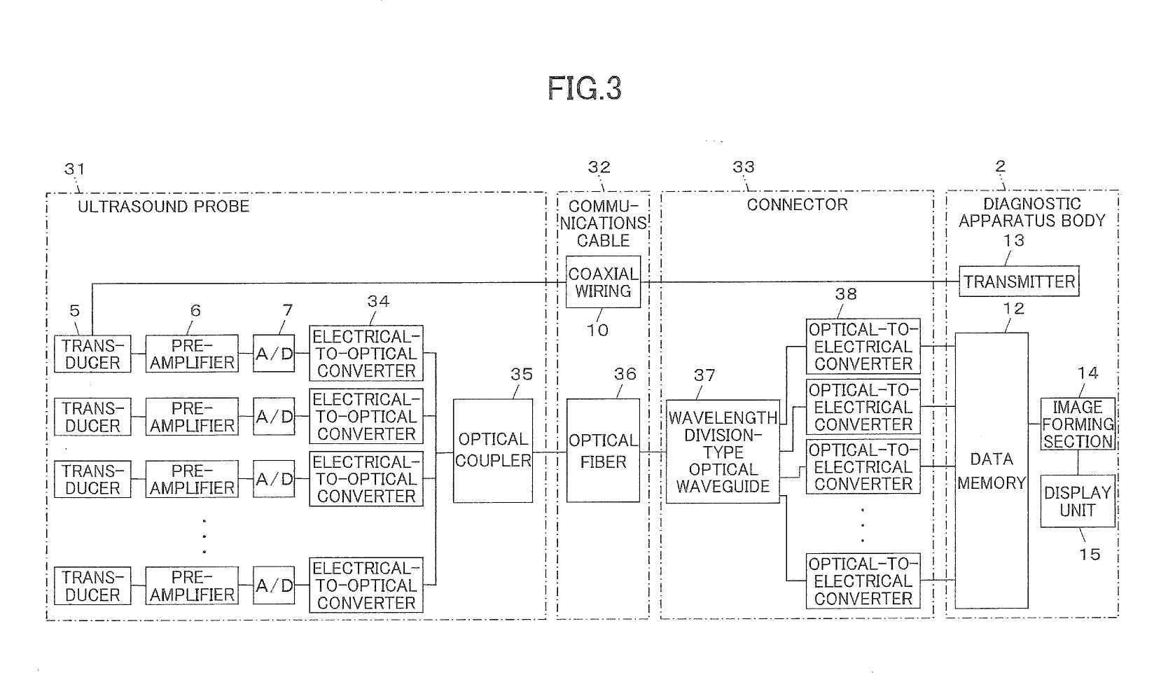

FIG. 3 illustrates the configuration of the ultrasound diagnostic apparatus according to Embodiment 3. In the ultrasound diagnostic apparatus of FIG. 3, the ultrasound probe 1, the communications cable 3 and the connector 4 in Embodiment 1 as shown in FIG. 1 are replaced by an ultrasound probe 31, a communications cable 32 and a connector 33, respectively. The ultrasound probe 31 does not have the electrical-to-optical converters 8 of the ultrasound probe 1 in Embodiment 1 but electrical-to-optical converters 34 correspondingly connected with the A/D converters 7, with the electrical-to-optical converters 34 being also connected to an optical coupler 35. The communications cable 32 has a single optical fiber 36 connected with the optical coupler 35 of the ultrasound probe 31, instead of the optical fibers 9 of the communications cable 3 in Embodiment 1. The connector 33 has a wavelength division-type optical waveguide 37 connected with the optical fiber 36, instead of the optical-to-electrical converters 11 of the connector 4 in Embodiment 1, with the optical waveguide 37 being also connected to optical-to-electrical converters 38 corresponding to the electrical-to-optical converters 34. The optical-to-electrical converters 38 of the connector 33 are each connected to the data memory 12 of the diagnostic apparatus body 2.

The electrical-to-optical converters 34 convert reception signals inputted therein as electric signals into optical signals with wavelengths different among the converters 34. The optical coupler 35 combines optical signals with different wavelengths resulting from the conversion by the electrical-to-optical converters 34 together into a composite optical signal to output it to the optical fiber 36 of the communications cable 32. The wavelength division-type optical waveguide 37 subjects the optical signal as transmitted from the optical coupler 35 via the optical fiber 36 to wavelength division and distributes optical signals obtained with different wavelengths among the optical-to-electrical converters 38 corresponding to the electrical-to-optical converters 34 according to their respective wavelengths. The optical-to-electrical converters 38 convert the optical signals as inputted from the wavelength division-type optical waveguide 37 into electric signals and then output them to the data memory 12 of the diagnostic apparatus body 2.

Similar to Embodiment 1, the reception signals as outputted from the transducer array 5 are amplified by the preamplifiers 6 and digitized by the A/D converters 7. The electrical-to-optical converters 34 convert the digitized reception signals as electric signals into optical signals with wavelengths different among the converters 34. The optical signals thus made distinguishable from one another are outputted from the individual electrical-to-optical converters 34 to the optical coupler 35, and combined together by the optical coupler 35. The obtained composite reception signal is transmitted from the optical coupler 35 to the wavelength division-type optical waveguide 37 via the optical fiber 36 of the communications cable 32, and subjected to wavelength division by the waveguide 37. The wavelength division yields reception signals with different wavelengths, which are distributed by the wavelength division-type optical waveguide 37 among the optical-to-electrical converters 38 corresponding to the electrical-to-optical converters 34 according to their respective wavelengths. The optical-to-electrical converters 38 convert the reception signals as optical signals into electric signals, and the reception signals as converted into electric signals are sequentially stored in the data memory 12 of the diagnostic apparatus body 2 as reception data.

According to Embodiment 3, the communications cable is reduced in thickness so as to make the cable easier to handle.

It is also possible to divide a plurality of electrical-to-optical converters 34 into two or more groups and connect each group of electrical-to-optical converters 34 to one optical fiber 36 through an optical coupler 35, so as to transmit optical signals group by group. Such configuration allows the communications cable 32 to be changed in number of optical fibers 36 therein depending on the situation.

### Embodiment 4

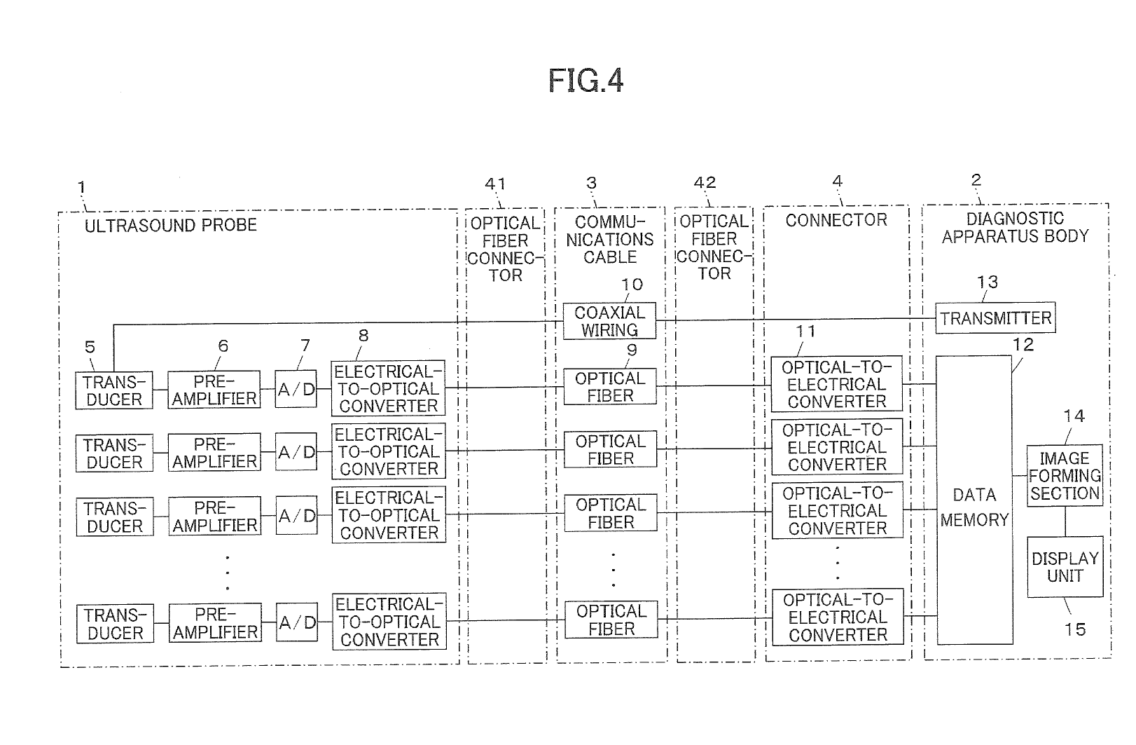

The ultrasound probe 1 and the connector 4 used in Embodiment 1 are integrally connected with the communications cable 3, to which the present invention is not limited. As shown in FIG. 4, for instance, the communications cable 3 may be connected with each of the ultrasound probe 1 and the connector 4 in a detachable manner.

The ultrasound diagnostic apparatus of FIG. 4 additionally has an optical fiber connector 41 provided between the ultrasound probe 1 and the communications cable 3 in Embodiment 1 as shown in FIG. 1, and an optical fiber connector 42 provided between the communications cable 3 and the connector 4. The electrical-to-optical converters 8 of the ultrasound probe 1 and the optical fibers 9 of the communications cable 3 are detachably connected with each other through the optical fiber connector 41, and the optical fibers 9 of the communications cable 3 and the optical-to-electrical converters 11 of the connector 4 are detachably connected with each other through the optical fiber connector 42.

According to Embodiment 4 in which the communications cable 3 is detachably connected with both the ultrasound probe 1 and the connector 4 through the optical fiber connectors 41 and 42, the communications cable 3 only needs to be changed for a further use of the ultrasound probe 1 in ultrasonic diagnosis if the optical fibers 9 having a lower durability than the ultrasound probe 1 or the connector 4 are damaged.

### Embodiment 5

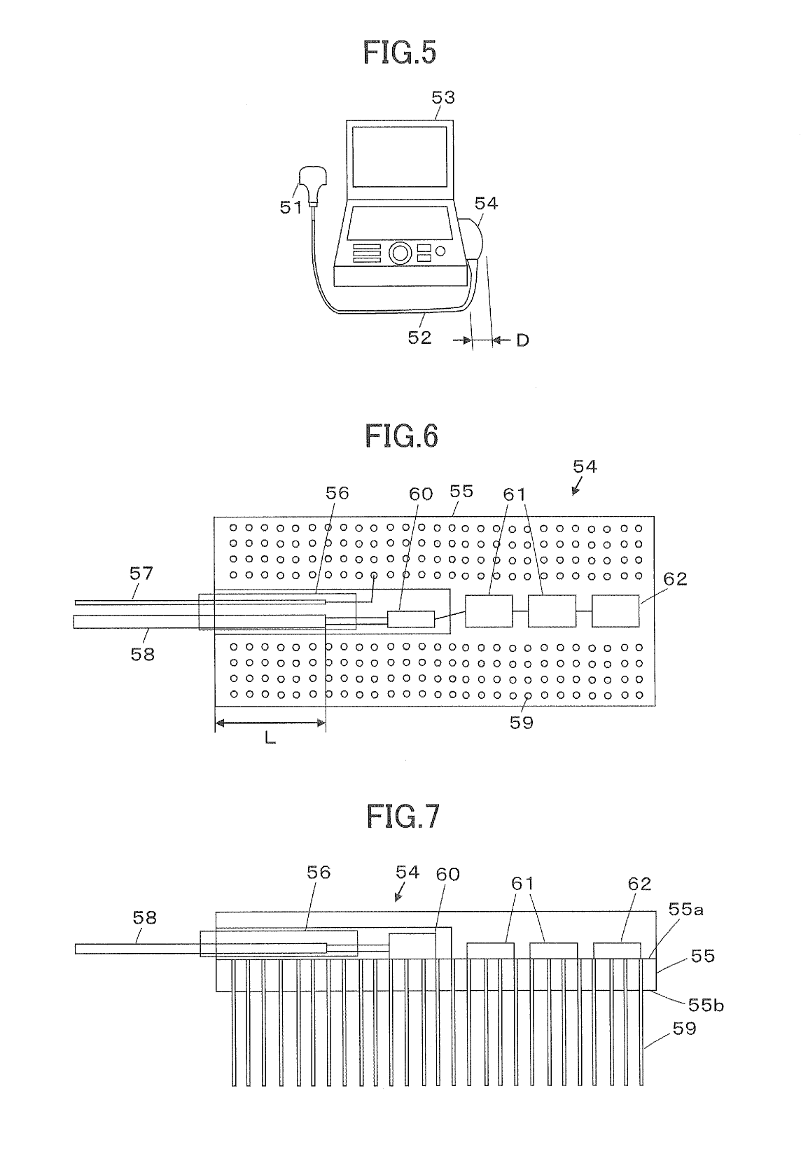

In the ultrasound diagnostic apparatus according to any of Embodiments 1 through 4, a connector 54 adapted to detachably connect a communications cable 52, which is connected in advance with an ultrasound probe 51, with a diagnostic apparatus body 53 may be so provided on a lateral face of the apparatus body 53 as to follow the lateral face, as shown in FIG. 5.

As an example, the connector used in the ultrasound diagnostic apparatus according to Embodiment 2 may have such a configuration as shown in FIG. 6. The connector 54 includes a connector board 55 approximately measuring 10 cm×5 cm, for instance, and is configured in a pigtail structure. In other words, the connector 54 has a cable connector 56 provided on one face 55a of the connector board 55, and the cable connector 56 receives therein one end of a coaxial wiring 57 and one end of an optical fiber 58, both extending from the communications cable 52, by a specified length L along the face 55a of the connector board 55 so as to secure them to the connector board 55. The connector 54 also has connector pins 59, which are uprightly provided at the other face 55b of the connector board 55 so that they may pierce through the connector board, as shown in FIG. 7. The cable connector 56 secures the tip portion of the optical fiber 58 as received therein by a specified length L to the connector board 55 while positioning the optical fiber 58 with such clearance as allowing the optical fiber 58 to be moved with no damage in the vicinity of a receptacle on the connector board 55. The optical fiber 58 is thus connected to the connector board 55 so as to obtain the connected structure which is hard to damage.

On the face 55a of the connector board 55, an optical-to-electrical converter 60 connected with the optical fiber 58, and a serial-to-parallel converter 62 connected with the optical-to-electrical converter 60 through an amplifier 61 (transimpedance amplifier, limiting amplifier or the like) are mounted. The serial-to-parallel converter 62 is connected to the connector pins 59 provided at the face 55b of the connector board 55, that is to say, the optical fiber 58 is connected to the connector pins 59 through the devices as above. The signal line dedicated to transmission signals that extends from the coaxial wiring 57 is also connected with the connector pins 59.

The communications cable 52 and the diagnostic apparatus body 53 are connected with each other by inserting the connector pins 59 uprightly provided on the connector board 55 into the diagnostic apparatus body 53. In consequence, the connector board 55 with the communications cable 52 extending therefrom is so provided on a lateral face of the diagnostic apparatus body 53 as to follow the lateral face.

According to Embodiment 5 in which the connector 54 is configured by arranging the optical fiber 58 along one face of the connector board 55 and providing the connector pits uprightly at the other face, the connector 54 having a smaller width D can be used. In addition, since the communications cable 52 is so positioned as to follow the lateral face of the diagnostic apparatus body 53, damage to the optical fiber 58 due to contact with the communications cable 52 is prevented. Moreover, plugging the optical fiber 58 into the connector board 55 by a specified length L allows a connected structure with suppressed damage to the optical fiber 58.

What is claimed is:

1. An ultrasound diagnostic apparatus, comprising:
an ultrasound probe adapted to transmit an ultrasonic beam from a transducer array toward a subject and receive an ultrasonic echo from the subject on the transducer array;
a diagnostic apparatus body adapted to generate an ultrasound image based on reception signals outputted from the transducer array of the ultrasound probe that has received an ultrasonic echo from the subject;
a communications cable adapted to connect the ultrasound probe and the diagnostic apparatus body with each other;
an apparatus body-side connector adapted to detachably connect one end of the communications cable with the diagnostic apparatus body;
a plurality of analog-to-digital converters contained in the ultrasound probe and adapted to process the reception signals outputted from the transducer array;
an electrical-to-optical conversion unit contained in the ultrasound probe and adapted to convert the reception signals having been processed by the plurality of analog-to-digital converters into optical signals;
an optical fiber provided in the communications cable and adapted to transmit the reception signals converted by the electrical-to-optical conversion unit into the optical signals; and
an optical-to-electrical conversion unit contained in the apparatus body-side connector and adapted to convert the reception signals transmitted by the optical fiber as the optical signals into electric signals, with the diagnostic apparatus body generating an ultrasound image based on the reception signals converted by the optical-to-electrical conversion unit into the electric signals.

2. The ultrasound diagnostic apparatus according to claim 1, further comprising:
a parallel-to-serial converter connected between said plurality of analog-to-digital converters and said electrical-to-optical conversion unit in said ultrasound probe, and adapted to subject the reception signals from the plurality of analog-to-digital converters to conversion from parallel data into serial data and then transmit them to the electrical-to-optical conversion unit; and
a serial-to-parallel converter connected downstream of said optical-to-electrical conversion unit in said apparatus body-side connector, and adapted to subject the reception signals from the optical-to-electrical conversion unit to conversion from serial data into parallel data and then transmit them to the diagnostic apparatus body.

3. The ultrasound diagnostic apparatus according to claim 1,
wherein said electrical-to-optical conversion unit includes a plurality of electrical-to-optical converters corresponding to said plurality of analog-to-digital converters, and said optical-to-electrical conversion unit includes a plurality of optical-to-electrical converters corresponding to the plurality of electrical-to-optical converters, and
wherein the ultrasound diagnostic apparatus further comprises:
an optical coupler adapted to combine the optical signals resulting from conversion by the plurality of electrical-to-optical converters together into composite optical signals and transmit the composite optical signals to said optical fiber; and
a wavelength division-type optical waveguide adapted to divide the composite optical signals transmitted via the optical fiber into optical signals with different wavelengths and feed the optical signals with different wavelengths to the plurality of optical-to-electrical converters, respectively.

4. The ultrasound diagnostic apparatus according to claim 1, wherein said apparatus body-side connector includes a connector board, and is adapted to receive one end of said optical fiber by a specified length along one face of the connector board and have connector pins uprightly provided at another face of the connector board.

5. The ultrasound diagnostic apparatus according to claim 2, wherein said apparatus body-side connector includes a connector board, and is adapted to receive one end of said optical fiber by a specified length along one face of the connector board and have connector pins uprightly provided at another face of the connector board.

6. The ultrasound diagnostic apparatus according to claim 3, wherein said apparatus body-side connector includes a connector board, and is adapted to receive one end of said optical fiber by a specified length along one face of the connector board and have connector pins uprightly provided at another face of the connector board.

7. The ultrasound diagnostic apparatus according to claim 4, wherein a cable connector for a signal line dedicated to transmission signals, said optical-to-electrical conversion unit, and an amplifier are mounted on one face of said connector board along with said optical fiber.

8. The ultrasound diagnostic apparatus according to claim 2,
wherein said apparatus body-side connector includes a connector board, and is adapted to receive one end of said optical fiber by a specified length along one face of the connector board and have connector pins uprightly provided at another face of the connector board, and
wherein a cable connector for a signal line dedicated to transmission signals, said optical-to-electrical conversion unit, an amplifier, and said serial-to-parallel converter are mounted on one face of the connector board along with the optical fiber.

9. The ultrasound diagnostic apparatus according to claim 1, wherein said communications cable is detachably connected with said ultrasound probe through a first optical fiber connector, and with said apparatus body-side connector through a second optical fiber connector.

10. The ultrasound diagnostic apparatus according to claim 2, wherein said communications cable is detachably connected with said ultrasound probe through a first optical fiber connector, and with said apparatus body-side connector through a second optical fiber connector.

11. The ultrasound diagnostic apparatus according to claim 3, wherein said communications cable is detachably connected with said ultrasound probe through a first optical fiber connector, and with said apparatus body-side connector through a second optical fiber connector.

12. The ultrasound diagnostic apparatus according to claim 4, wherein said communications cable is detachably connected with said ultrasound probe through a first optical fiber connector, and with said apparatus body-side connector through a second optical fiber connector.

13. The ultrasound diagnostic apparatus according to claim 5, wherein said communications cable is detachably connected with said ultrasound probe through a first optical fiber connector, and with said apparatus body-side connector through a second optical fiber connector.

14. The ultrasound diagnostic apparatus according to claim 6, wherein said communications cable is detachably connected with said ultrasound probe through a first optical fiber connector, and with said apparatus body-side connector through a second optical fiber connector.

15. The ultrasound diagnostic apparatus according to claim 7, wherein said communications cable is detachably connected with said ultrasound probe through a first optical fiber connector, and with said apparatus body-side connector through a second optical fiber connector.

16. The ultrasound diagnostic apparatus according to claim 8, wherein said communications cable is detachably connected with said ultrasound probe through a first optical fiber connector, and with said apparatus body-side connector through a second optical fiber connector.
